(19) United States
(12) Patent Application Publication
(10) Pub. No.: US 2024/0053256 A1
(43) Pub. Date: Feb. 15, 2024

(54) OPTICAL SENSOR DEVICE

(71) Applicant: Mitsubishi Electric Corporation, Tokyo (JP)

(72) Inventors: Junya NISHIOKA, Tokyo (JP); Takanori YAMAUCHI, Tokyo (JP); Naoki SUZUKI, Tokyo (JP)

(73) Assignee: Mitsubishi Electric Corporation, Tokyo (JP)

(21) Appl. No.: 18/379,584

(22) Filed: Oct. 12, 2023

Related U.S. Application Data
(63) Continuation of application No. PCT/JP2021/023157, filed on Jun. 18, 2021.

(51) Int. Cl. G01N 21/17

(52) U.S. Cl. CPC G01N 21/17

(57) ABSTRACT

A signal processing device in an optical sensor device further calculates first frequency variation reference signal data serving as a reference for frequency variation of light output from a wavelength swept light source on the basis of an internal reception signal converted into a digital signal by an analog-to-digital converter, a digital-to-analog converter converts the first frequency variation reference signal data calculated by the signal processing device into an analog signal to generate a first frequency variation reference signal as a first clock signal, and the analog-to-digital converter samples a reception signal acquired by an optical heterodyne receiver in synchronization with the first frequency variation reference signal generated by the digital-to-analog converter.

### CROSS REFERENCE TO RELATED APPLICATION

This application is a Continuation of PCT International Application No. PCT/JP2021/023157, filed on Jun. 18, 2021, all of which is hereby expressly incorporated by reference into the present application.

### TECHNICAL FIELD

The present disclosure relates to an optical sensor device.

### BACKGROUND ART

A swept source-optical coherence tomography (SS-OCT) adopting a wavelength scanning interferometry system branches wavelength swept light whose frequency changes with the lapse of time into signal light and reference light. The SS-OCT emits branched signal light toward a measurement target, receives the signal light reflected by the measurement target, and acquires a beat signal by causing the received signal light to interfere with the branched reference light and generating interference light. The SS-OCT measures the distance from the light source to the measurement target by measuring the frequency of the acquired beat signal.

When the SS-OCT as described above sweeps the frequency of light in a wide band, the temporal change of the frequency of the wavelength swept light does not exhibit ideal linearity and exhibits nonlinearity, so that the above-described distance resolution is deteriorated. Therefore, the optical distance measuring device described in Patent Literature 1 compensates for such nonlinearity of the wavelength swept light. More specifically, the optical distance measuring device compensates for nonlinearity of the wavelength swept light by performing regression analysis on the beat signal on the basis of a known frequency modulation waveform by digital signal processing using a laser light source having a known frequency modulation waveform.

### CITATION LIST

### Patent Literature

Patent Literature 1: WO 2018/230474 A

### SUMMARY OF INVENTION

### Technical Problem

However, in the method of Patent Literature 1, a regression analysis for compensating for nonlinearity of the wavelength swept light is required for each measurement, and there is a problem that a signal processing load increases.

The present disclosure has been made to solve the above-described problems, and provides a technique for reducing a signal processing load caused by compensating for nonlinearity of wavelength swept light.

### Solution to Problem

An optical sensor device according to the present disclosure includes: a wavelength swept light source to output light whose frequency changes with lapse of time; an optical brancher to branch light output from the wavelength swept light source 1 into signal light and local oscillation light; an optical sensor head to emit the signal light branched by the optical brancher toward a measurement target and receive reflected light reflected by the measurement target; an optical heterodyne receiver to multiplex the local oscillation light branched by the optical brancher and the reflected light received by the optical sensor head, and photoelectrically convert the multiplexed light to acquire a reception signal as an electric signal; an analog-to-digital converter to convert the reception signal acquired by the optical heterodyne receiver into a digital signal by sampling the reception signal; a first digital-to-analog converter to generate a first clock signal of the analog-to-digital converter; a phase-locked loop to generate a second clock signal of the analog-to-digital converter; and a signal processor to calculate measurement data related to the measurement target on a basis of the reception signal converted into the digital signal by the analog-to-digital converter, wherein the optical heterodyne receiver multiplexes the local oscillation light branched by the optical brancher and internal reflected light obtained by internally reflecting the signal light branched by the optical brancher, and photoelectrically converts the multiplexed light to further acquire an internal reception signal as an electric signal, the analog-to-digital converter further converts the internal reception signal acquired by the optical heterodyne receiver into a digital signal by sampling the internal reception signal, the signal processor further calculates first frequency variation reference signal data serving as a reference for frequency variation of light output from the wavelength swept light source on a basis of the internal reception signal converted into a digital signal by the analog-to-digital converter, the first digital-to-analog converter generates a first frequency variation reference signal as the first clock signal by converting the first frequency variation reference signal data calculated by the signal processor into an analog signal, the analog-to-digital converter samples the reception signal acquired by the optical heterodyne receiver in synchronization with the first frequency variation reference signal generated by the first digital-to-analog converter or, samples the internal reception signal acquired by the optical heterodyne receiver in synchronization with the second clock signal generated by the phase-locked loop.

### Advantageous Effects of Invention

According to the present disclosure, a signal processing load caused by compensating for nonlinearity of wavelength swept light is reduced.

### BRIEF DESCRIPTION OF DRAWINGS

FIG. 1 is a block diagram illustrating a configuration of an optical sensor device according to a first embodiment.

FIG. 2 is a graph for describing a specific example of signal processing by an optical sensor in a case where a frequency of wavelength swept light exhibits linearity.

FIG. 3 is a graph for explaining a specific example of signal processing by the optical sensor device in a case where nonlinearity is not compensated for.

FIG. 4 is a graph for describing a specific example of signal processing for internal reflected light by the optical sensor device according to the first embodiment.

FIG. 5 is a graph for describing a specific example of signal processing for reflected light by the optical sensor device according to the first embodiment.

FIG. 6 is a block diagram illustrating a configuration of an optical sensor device according to a second embodiment.

FIG. 7 is a graph for describing a specific example of signal processing for internal reflected light by the optical sensor device according to the second embodiment.

FIG. 8 is a graph for describing a specific example of signal processing for reflected light by the optical sensor device according to the second embodiment.

FIG. 9 is a block diagram illustrating a configuration of an optical sensor device according to a third embodiment.

FIG. 10 is a graph for describing a specific example of a method for separating a reception signal and an internal reception signal by the optical sensor device according to the third embodiment.

FIG. 11 is a block diagram illustrating a configuration of an optical sensor device according to a fourth embodiment.

FIG. 12 is a graph illustrating a time change in a frequency of an internal reception signal acquired by an optical heterodyne receiver multiplexing local oscillation light and internal reflected light and photoelectrically converting the multiplexed light in a specific example of the fourth embodiment.

FIG. 13A is a block diagram illustrating a hardware configuration that implements functions of a signal processing device according to the first to fourth embodiments. FIG. 13B is a block diagram illustrating a hardware configuration for executing software for implementing the functions of the signal processing device according to the first to fourth embodiments.

### DESCRIPTION OF EMBODIMENTS

In order to explain the present disclosure in more detail, embodiments for carrying out the present disclosure will be described below with reference to the accompanying drawings.

### First Embodiment

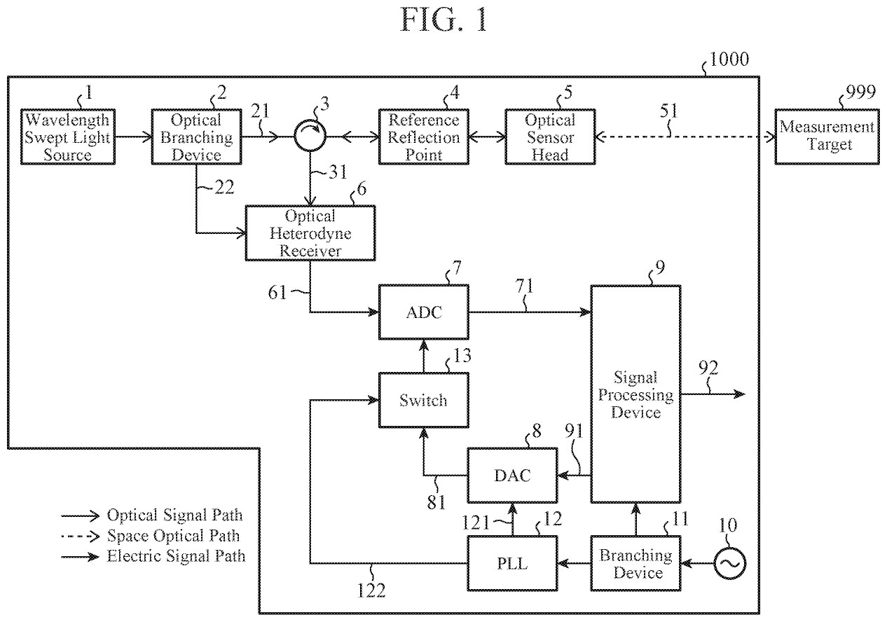

FIG. 1 is a block diagram illustrating a configuration of an optical sensor device 1000 according to a first embodiment. As illustrated in FIG. 1, the optical sensor device 1000 includes a wavelength swept light source 1, an optical branching device 2, an optical circulator 3, a reference reflection point 4, an optical sensor head 5, an optical heterodyne receiver 6, an analog-to-digital converter 7 (ADC), a digital-to-analog converter 8 (DAC) (first digital-to-analog converter), a signal processing device 9, a reference clock 10, a branching device 11, a phase-locked loop 12 (PLL), and a switch 13.

The wavelength swept light source 1 outputs light (wavelength swept light) whose frequency changes with lapse of time to the optical branching device 2. That is, the wavelength swept light source 1 performs frequency sweep (wavelength sweep). In other words, the wavelength swept light source 1 outputs light whose wavelength changes with lapse of time to the optical branching device 2.

For example, as the wavelength swept light source 1, a laser light source capable of wavelength control by controlling a resonator length, a laser light source whose wavelength changes according to an injection current amount, or the like can be used. For example, the wavelength swept light source 1 may output light that alternately repeats a continuous triangular wave of up chirp and down chirp by performing frequency sweeping, may output light that repeats a sawtooth wave of up chirp, may output light that repeats a sawtooth wave of down chirp, or may output a chirp pulse signal of pulsed up chirp or down chirp.

The optical branching device 2 branches the light output from the wavelength swept light source into signal light and local oscillation light. The optical branching device 2 outputs the branched signal light to the optical circulator 3 and outputs the branched local oscillation light to the optical heterodyne receiver 6 (22 in FIG. 1).

The optical circulator 3 outputs the signal light branched by the optical branching device 2 to the reference reflection point 4.

The reference reflection point 4 internally reflects the signal light by partially reflecting the signal light branched by the optical branching device 2. More specifically, in the first embodiment, the reference reflection point 4 internally reflects the signal light output from the optical circulator 3 by partially reflecting the signal light. The internal reflected light internally reflected by the reference reflection point 4 is output to the optical heterodyne receiver 6 via the optical circulator 3. The signal light having passed through the reference reflection point 4 is output to the optical sensor head 5. Examples of the reference reflection point 4 include a partial reflection mirror or a connector end surface.

The optical sensor head 5 emits signal light (51 in FIG. 1) branched by the optical branching device 2 toward a measurement target 999, and receives reflected light (51 in FIG. 1) reflected by the measurement target 999. More specifically, in the first embodiment, the optical sensor head 5 emits signal light (51 in FIG. 1) having passed through the reference reflection point 4 toward the measurement target 999, and receives reflected light (51 in FIG. 1) reflected by the measurement target 999. The optical sensor head 5 outputs the received reflected light to the optical heterodyne receiver 6 via the reference reflection point 4 and the optical circulator 3 (31 in FIG. 1).

Note that, ss described above, the optical circulator 3 outputs signal light (21 in FIG. 1) input from the optical branching device 2 side to the reference reflection point 4, and outputs the reflected light or the internal reflected light (31 in FIG. 1) input from the reference reflection point 4 side to the optical heterodyne receiver 6.

The optical heterodyne receiver 6 multiplexes local oscillation light (22 in FIG. 1) branched by the optical branching device 2 and reflected light (31 in FIG. 1) received by the optical sensor head 5, and photoelectrically converts the multiplexed light to acquire a reception signal (beat signal) as an electric signal. That is, the optical heterodyne receiver 6 performs heterodyne processing on the local oscillation light (22 in FIG. 1) branched by the optical branching device 2 and the reflected light (31 in FIG. 1) received by the optical sensor head 5. Note that the optical heterodyne receiver 6 photoelectrically converts the multiplexed light using, for example, a photodiode (PD).

On the other hand, the optical heterodyne receiver 6 multiplexes the local oscillation light (22 in FIG. 1) branched by the optical branching device 2 and the internal reflected light (31 in FIG. 1) obtained by internally reflecting the signal light branched by the optical branching device 2, and photoelectrically converts the multiplexed light to further acquire an internal reception signal as an electric signal. More specifically, the optical heterodyne receiver 6 multiplexes the local oscillation light (22 in FIG. 1) branched by the optical branching device 2 and the internal reflected light (31 in FIG. 1) reflected by the reference reflection point 4, and photoelectrically converts the multiplexed light to further acquire an internal reception signal as an electric signal. The optical heterodyne receiver 6 outputs the acquired reception signal and internal reception signal (61 in FIG. 1) to the analog-to-digital converter 7.

The reference clock 10 generates a reference clock signal. The reference clock 10 outputs the generated reference clock signal to the branching device 11. The branching device 11 branches the reference clock signal generated by the reference clock 10 into the signal processing device 9 and the phase-locked loop 12.

The phase-locked loop 12 (PLL) generates a second clock signal for the analog-to-digital converter 7. More specifically, in the first embodiment, the phase-locked loop 12 generates the second clock signal of the analog-to-digital converter 7 in synchronization with the reference clock signal branched by the branching device 11. The phase-locked loop 12 outputs the generated second clock signal (121 in FIG. 1) to the digital-to-analog converter 8, and outputs the generated second clock signal (122 in FIG. 1) to the switch 13.

The digital-to-analog converter 8 (DAC) generates a first clock signal of the analog-to-digital converter 7. More specifically, the digital-to-analog converter 8 generates the first clock signal of the analog-to-digital converter 7 in synchronization with the second clock signal generated by the phase-locked loop 12. The digital-to-analog converter 8 outputs the generated first clock signal (81 in FIG. 1) to the switch 13. Details of the first clock signal will be described later.

Note that, as described above, in the first embodiment, a configuration in which the digital-to-analog converter 8 generates the first clock signal of the analog-to-digital converter 7 in synchronization with the second clock signal generated by the phase-locked loop 12 will be described. However, the optical sensor device 1000 may further include a circuit that generates a clock, and the digital-to-analog converter 8 may generate the first clock signal of the analog-to-digital converter 7 in synchronization with the clock generated by the circuit. That is, the frequency of the first clock signal and the frequency of the second clock signal do not need to be synchronized.

The switch 13 switches the clock signal of the analog-to-digital converter 7 to either the first clock signal generated by the digital-to-analog converter 8 or the second clock signal generated by the phase-locked loop 12. For example, when the optical sensor device 1000 acquires first frequency variation reference signal data to be described later, the switch 13 switches the clock signal of the analog-to-digital converter 7 to the second clock signal generated by the phase-locked loop 12. For example, when the optical sensor device 1000 acquires measurement data related to the measurement target 999 to be described later, the switch 13 switches the clock signal of the analog-to-digital converter 7 to the first clock signal generated by the digital-to-analog converter 8.

The analog-to-digital converter 7 converts the internal reception signal acquired by the optical heterodyne receiver 6 into a digital signal by sampling the internal reception signal. More specifically, in the first embodiment, the analog-to-digital converter 7 samples the internal reception signal acquired by the optical heterodyne receiver 6 in synchronization with the second clock signal generated by the phase-locked loop 12. More specifically, in the first embodiment, the analog-to-digital converter 7 samples the internal reception signal acquired by the optical heterodyne receiver 6 in synchronization with the second clock signal switched by the switch 13. The analog-to-digital converter 7 outputs the internal reception signal (71 in FIG. 1) converted into the digital signal to the signal processing device 9.

The signal processing device 9 calculates first frequency variation reference signal data serving as a reference for the frequency variation of the light output from the wavelength swept light source 1 on the basis of the internal reception signal converted into the digital signal by the analog-to-digital converter 7.

More specifically, in the first embodiment, the signal processing device 9 calculates the first frequency variation reference signal data on the basis of the internal reception signal converted into a digital signal by the analog-to-digital converter 7 in synchronization with the reference clock signal branched by the branching device 11. The signal processing device 9 outputs the calculated first frequency variation reference signal data to the digital-to-analog converter 8 (91 in FIG. 1). More specifically, the signal processing device 9 stores the calculated first frequency variation reference signal data in a memory (not illustrated), and the memory outputs the stored first frequency variation reference signal data to the digital-to-analog converter 8. Details of the first frequency variation reference signal data will be described later.

The digital-to-analog converter 8 generates a first frequency variation reference signal as the first clock signal by converting the first frequency variation reference signal data calculated by the signal processing device 9 into an analog signal. More specifically, in the first embodiment, the digital-to-analog converter 8 generates the first frequency variation reference signal as the first clock signal by converting the first frequency variation reference signal data calculated by the signal processing device 9 into an analog signal in synchronization with the second clock signal generated by the phase-locked loop 12. The digital-to-analog converter 8 outputs the generated first frequency variation reference signal to the switch 13.

The analog-to-digital converter 7 (ADC) further converts the reception signal acquired by the optical heterodyne receiver 6 into a digital signal by sampling the reception signal. More specifically, the analog-to-digital converter 7 samples the reception signal acquired by the optical heterodyne receiver 6 in synchronization with the first frequency variation reference signal generated by the digital-to-analog converter 8. More specifically, in the first embodiment, the analog-to-digital converter 7 samples the reception signal acquired by the optical heterodyne receiver 6 in synchronization with the first frequency variation reference signal switched by the switch 13. The analog-to-digital converter 7 outputs the reception signal (71 in FIG. 1) converted into the digital signal to the signal processing device 9.

The signal processing device 9 calculates measurement data related to the measurement target 999 on the basis of the reception signal converted into a digital signal by the analog-to-digital converter 7. The signal processing device 9 outputs the calculated measurement data to the outside of the device (92 in FIG. 1). Although not illustrated, the optical sensor device 1000 may further include a display device that displays the calculated measurement data as an image. Examples of the measurement data calculated by the signal processing device 9 include information indicating the distance from the optical sensor device 1000 to the measurement target 999, information indicating the position of the measurement target 999, or the like.

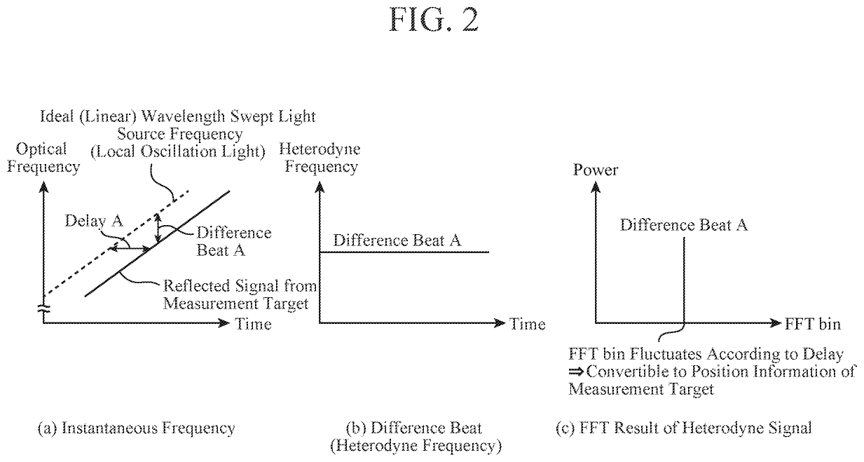

Hereinafter, a specific example of a method for compensating for nonlinearity of wavelength swept light by the optical sensor device 1000 according to the first embodiment will be described with reference to the drawings. First, for comparison, an example in which the frequency of the wavelength swept light exhibits linearity will be described. FIG. 2 is a graph for describing a specific example of signal processing by the optical sensor device 1000 in a case where the frequency of the wavelength swept light exhibits linearity. That is, in the specific example, the wavelength swept light source 1 outputs wavelength swept light (For example, linear up chirp or the like) exhibiting linearity.

(a) of FIG. 2 is a graph illustrating a time change (broken line) in the frequency of the local oscillation light branched by the optical branching device 2 and a time change (solid line) in the frequency of the reflected light received from the measurement target 999 by the optical sensor head 5. (b) of FIG. 2 is a graph illustrating a time change in the frequency (heterodyne frequency) of the reception signal (difference beat A) acquired by the optical heterodyne receiver 6 multiplexing the local oscillation light and the reflected light and photoelectrically converting the multiplexed light. (c) of FIG. 2 is a graph illustrating a frequency spectrum that is a result of fast Fourier transform (FFT) performed by the signal processing device 9 on the reception signal converted into the digital signal by the analog-to-digital converter 7.

As in the specific example, when the frequency of the wavelength swept light output from the wavelength swept light source 1 exhibits ideal linearity, the time delay A between the local oscillation light and the reflected light reflected by the measurement target 999 is constant as illustrated in (a) of FIG. 2, and the frequency of the difference beat A, which is a beat signal obtained by multiplexing the local oscillation light and the reflected light, is also constant as illustrated in (b) of FIG. 2. Therefore, as illustrated in (c) of FIG. 2, the frequency spectrum based on the difference beat A shows a sharp peak at a specific frequency. As a result, the signal processing device 9 can calculate the position information of the measurement target on the basis of the FFT bin number including the specific frequency.

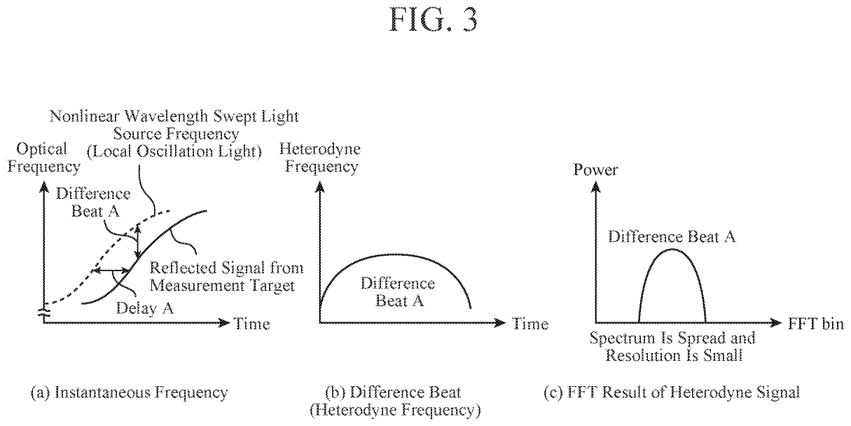

Next, for comparison, an example in which the frequency of the wavelength swept light exhibits nonlinearity, but the optical sensor device 1000 does not compensate for nonlinearity will be described. FIG. 3 is a graph for explaining a specific example of signal processing by the optical sensor device 1000 in a case of not compensating for nonlinearity. That is, in the specific example, the wavelength swept light source 1 outputs the wavelength swept light (For example, linear up chirp or the like) exhibiting nonlinearity. Further, in this specific example, it is assumed that the analog-to-digital converter 7 samples the reception signal acquired by the optical heterodyne receiver 6 in synchronization with the second clock signal generated by the phase-locked loop 12 without being synchronized with the first frequency variation reference signal generated by the digital-to-analog converter 8 as described above.

(a) of FIG. 3 is a graph illustrating a time change (broken line) in the frequency of the local oscillation light branched by the optical branching device 2 and a time change (solid line) in the frequency of the reflected light received from the measurement target 999 by the optical sensor head 5 in the specific example. (b) of FIG. 3 is a graph illustrating a time change in the frequency (heterodyne frequency) of the reception signal (difference beat A) acquired by the optical heterodyne receiver 6 multiplexing the local oscillation light and the reflected light and photoelectrically converting the multiplexed light in the specific example. (c) of FIG. 3 is a graph illustrating a frequency spectrum that is a result of fast Fourier transform (FFT) performed by the signal processing device 9 on the reception signal converted into the digital signal by the analog-to-digital converter 7 in the specific example.

As in the specific example, when the frequency of the wavelength swept light output from the wavelength swept light source 1 exhibits nonlinearity, as illustrated in (a) of FIG. 3, the frequency of the local oscillation light and the frequency of the reflected light reflected by the measurement target 999 each exhibit a curve, and the time delay A between the local oscillation light and the reflected light reflected by the measurement target 999 changes with lapse of time. Therefore, as illustrated in (b) of FIG. 3, the frequency of the difference beat A, which is a beat signal obtained by multiplexing them, also changes with lapse of time. Therefore, as illustrated in (c) of FIG. 3, the frequency spectrum based on the difference beat A spreads in the frequency axis direction, and the resolution of the position measurement of the measurement target decreases.

Next, a specific example of signal processing by the optical sensor device 1000 according to the first embodiment will be described. That is, a specific example of a configuration in which the frequency of the wavelength swept light exhibits nonlinearity and the optical sensor device 1000 compensates for nonlinearity will be described.

First, in a state where the reflected light reflected from the measurement target 999 is blocked, the optical heterodyne receiver 6 multiplexes the local oscillation light branched by the optical branching device 2 and the internal reflected light reflected by the reference reflection point 4, and photoelectrically converts the multiplexed light to acquire an internal reception signal as an electric signal. The analog-to-digital converter 7 converts the internal reception signal acquired by the optical heterodyne receiver 6 into a digital signal by sampling the internal reception signal in synchronization with the second clock signal (the second clock signal generated by the phase-locked loop 12) switched by the switch 13.

The signal processing device 9 calculates first frequency variation reference signal data on the basis of the internal reception signal converted into a digital signal by the analog-to-digital converter 7, and stores the first frequency variation reference signal data in a memory (not illustrated). For example, the signal processing device 9 calculates the instantaneous frequency of the internal reception signal by performing Hilbert transform on the internal reception signal converted into a digital signal by the analog-to-digital converter 7, and calculates the first frequency variation reference signal data by multiplying the calculated instantaneous frequency. More specifically, for example, the signal processing device 9 calculates the instantaneous frequency f_{ref}(t) of the internal reception signal by performing Hilbert transform on the internal reception signal converted into a digital signal by the analog-to-digital converter 7, and calculates the first frequency variation reference signal data of the frequency component Kf_{ref}(t) by multiplying the calculated instantaneous frequency f_{ref}(t) by K. Here, K is a positive integer.

The digital-to-analog converter 8 generates a first frequency variation reference signal as a first clock signal by converting first frequency variation reference signal data calculated by the signal processing device 9 and stored in a memory (not illustrated) into an analog signal.

In this specific example, the analog-to-digital converter 7 converts each of the internal reception signal and the reception signal acquired by the optical heterodyne receiver 6 into a digital signal by sampling each of the internal reception signal and the reception signal in synchronization with the first frequency variation reference signal generated by the digital-to-analog converter 8. Note that the internal reception signal here is acquired again by the optical heterodyne receiver 6. In addition, as described above, the reception signal here is acquired as an electric signal by the optical heterodyne receiver 6 multiplexing local oscillation light branched by the optical branching device 2 and reflected light received by the optical sensor head 5 and photoelectrically converting the multiplexed light.

In this specific example, the signal processing device 9 performs fast Fourier transform (FFT) on each of the internal reception signal and the reception signal converted into the digital signal by the analog-to-digital converter 7.

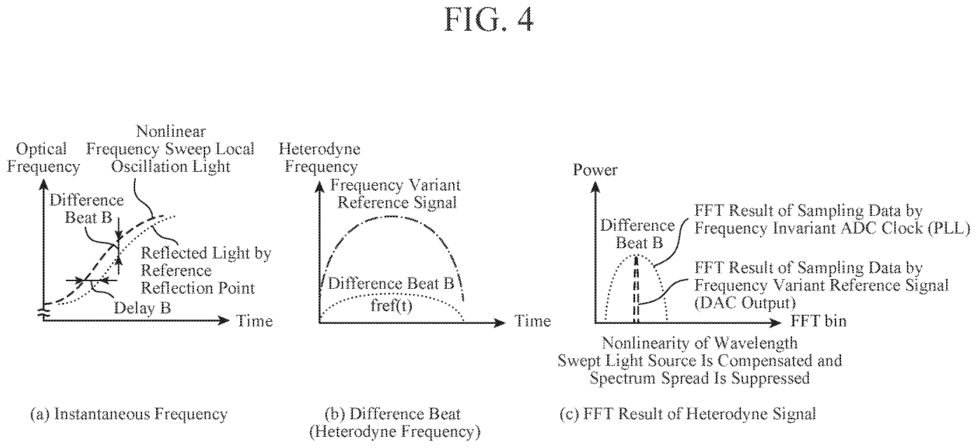

FIG. 4 is a graph for describing a specific example of signal processing for internal reflected light by optical sensor device 1000 according to the first embodiment. (a) of FIG. 4 is a graph illustrating a time change (broken line) in the frequency of the local oscillation light branched by the optical branching device 2 and a time change (dotted line) in the frequency of the internal reflected light reflected by the reference reflection point 4 in the specific example.

(b) of FIG. 4 is a graph illustrating a time change (dotted line) of the frequency (heterodyne frequency) of the internal reception signal (difference beat B) acquired by the optical heterodyne receiver 6 multiplexing the local oscillation light and the internal reflected light and photoelectrically converting the multiplexed light in the specific example. Note that an alternate long and short dash line in (b) of FIG. 4 indicates the first frequency variation reference signal.

(c) of FIG. 4 is a graph illustrating a frequency spectrum (broken line) that is a result of the fast Fourier transform of the internal reception signal performed by the signal processing device 9 in the specific example. Note that the internal reception signal here is acquired again by the optical heterodyne receiver 6 and converted into a digital signal by the analog-to-digital converter 7 sampling in synchronization with the first frequency variation reference signal described above. In addition, a dotted line in (c) of FIG. 4 indicates a frequency spectrum in a case where the analog-to-digital converter 7 converts the internal reception signal into a digital signal by sampling the internal reception signal in synchronization with the second clock signal of the phase-locked loop 12 described above.

As illustrated in (a) of FIG. 4, the frequency of the local oscillation light and the frequency of the internal reflected light reflected by the reference reflection point 4 each indicate a curve, and the time delay B between the local oscillation light and the internal reflected light changes with lapse of time. Therefore, as indicated by the dotted line in (b) of FIG. 4, the frequency of the difference beat B, which is a beat signal obtained by multiplexing them, also changes with lapse of time, similarly to the difference beat A in (b) of FIG. 3. However, in this specific example, the analog-to-digital converter 7 samples the internal reception signal in synchronization with the first frequency variation reference signal described above, thereby compensating for the nonlinearity of the wavelength swept light, so that the spread of the spectrum is suppressed as indicated by the broken line in (c) of FIG. 4.

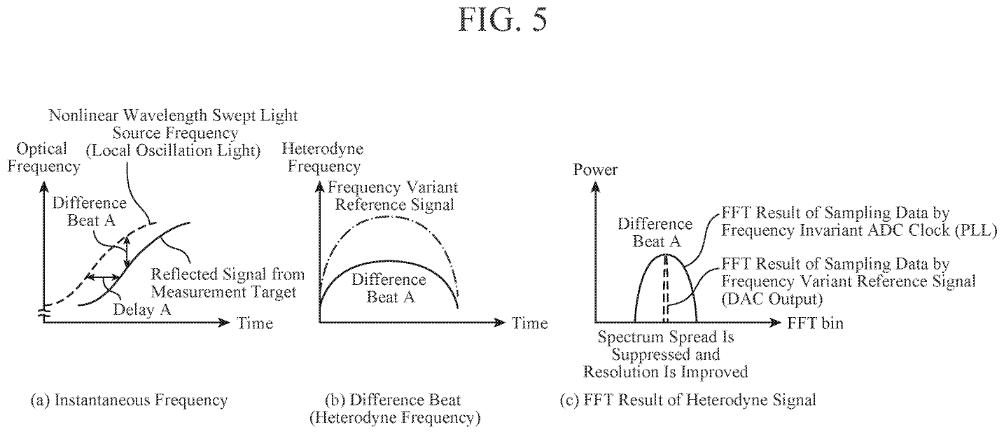

FIG. 5 is a graph for describing a specific example of signal processing for reflected light by the optical sensor device 1000 according to the first embodiment. (a) of FIG. 5 is a graph illustrating a time change (broken line) in the frequency of the local oscillation light branched by the optical branching device 2 and a time change (solid line) in the frequency of the reflected light received from the measurement target 999 by the optical sensor head 5 in the specific example.

(b) of FIG. 5 is a graph illustrating a time change (solid line) in the frequency (heterodyne frequency) of the reception signal (difference beat A) acquired by the optical heterodyne receiver 6 multiplexing the local oscillation light and the reflected light and photoelectrically converting the multiplexed light in the specific example. Note that an alternate long and short dash line in (b) of FIG. 5 indicates the first frequency variation reference signal.

(c) of FIG. 5 is a graph illustrating a frequency spectrum (broken line) that is a result of the fast Fourier transform of the reception signal performed by the signal processing device 9 in the specific example. Here, the reception signal is converted into a digital signal by sampling in synchronization with the first frequency variation reference signal by the analog-to-digital converter 7. In addition, a solid line in (c) of FIG. 5 indicates a frequency spectrum in a case where the analog-to-digital converter 7 converts the reception signal into a digital signal by sampling the reception signal in synchronization with the second clock signal of the phase-locked loop 12 described above.

As illustrated in (a) of FIG. 5, the frequency of the local oscillation light and the frequency of the reflected light received from the measurement target 999 by the optical sensor head 5 each indicate a curve, and the time delay A between the local oscillation light and the reflected light changes with lapse of time. Therefore, as indicated by the solid line in (b) of FIG. 5, the frequency of the difference beat A, which is a beat signal obtained by multiplexing them, also changes with lapse of time. However, in this specific example, the analog-to-digital converter 7 samples the reception signal in synchronization with the first frequency variation reference signal described above, thereby compensating for the nonlinearity of the wavelength swept light, so that the spread of the spectrum is suppressed as indicated by the broken line in (c) of FIG. 5. As a result, the signal processing device 9 can calculate the position information of the measurement target on the basis of the FFT bin number.

As described above, in the first embodiment, by adopting the configuration in which the sampling is performed with reference to the first frequency variation reference signal data calculated in advance on the basis of the internal reflected light, it is possible to implement a high-accuracy optical sensor device 1000 that is simple and has a reduced signal processing load at the time of measurement.

As described above, the optical sensor device 1000 according to the first embodiment includes: the wavelength swept light source 1 to output light whose frequency changes with lapse of time; the optical branching device 2 to branch light output from the wavelength swept light source 1 into signal light and local oscillation light; the optical sensor head 5 to emit the signal light branched by the optical branching device 2 toward a measurement target and receive reflected light reflected by the measurement target; the optical heterodyne receiver 6 to multiplex the local oscillation light branched by the optical branching device 2 and the reflected light received by the optical sensor head 5, and photoelectrically convert the multiplexed light to acquire a reception signal as an electric signal; the analog-to-digital converter 7 to convert the reception signal acquired by the optical heterodyne receiver 6 into a digital signal by sampling the reception signal; the digital-to-analog converter 8 to generate a first clock signal of the analog-to-digital converter 7; and the signal processing device 9 to calculate measurement data related to the measurement target on the basis of the reception signal converted into the digital signal by the analog-to-digital converter 7, in which the optical heterodyne receiver 6 multiplexes the local oscillation light branched by the optical branching device 2 and internal reflected light obtained by internally reflecting the signal light branched by the optical branching device 2, and photoelectrically converts the multiplexed light to further acquire an internal reception signal as an electric signal, the analog-to-digital converter 7 further converts the internal reception signal acquired by the optical heterodyne receiver 6 into a digital signal by sampling the internal reception signal, the signal processing device 9 further calculates first frequency variation reference signal data serving as a reference for frequency variation of light output from the wavelength swept light source 1 on the basis of the internal reception signal converted into a digital signal by the analog-to-digital converter 7, the digital-to-analog converter 8 generates a first frequency variation reference signal as the first clock signal by converting the first frequency variation reference signal data calculated by the signal processing device 9 into an analog signal, and the analog-to-digital converter 7 samples the reception signal acquired by the optical heterodyne receiver 6 in synchronization with the first frequency variation reference signal generated by the digital-to-analog converter 8.

According to the above configuration, the nonlinearity of the wavelength swept light can be compensated by sampling the reception signal derived from the reflected light from the measurement target in synchronization with the first frequency variation reference signal derived from the internal reception signal. This eliminates the need for signal processing for compensating for nonlinearity of the wavelength swept light for each measurement, so that a signal processing load caused by compensating for nonlinearity of the signal processing wavelength swept light can be reduced.

### Second Embodiment

In the first embodiment, the configuration in which the waveform of the wavelength swept light output from the wavelength swept light source 1 does not change has been described. However, in a case where the waveform of the wavelength swept light changes, the resolution of the position measurement of the measurement target decreases. Therefore, in the second embodiment, a configuration for compensating for nonlinearity of wavelength swept light whose waveform changes will be described.

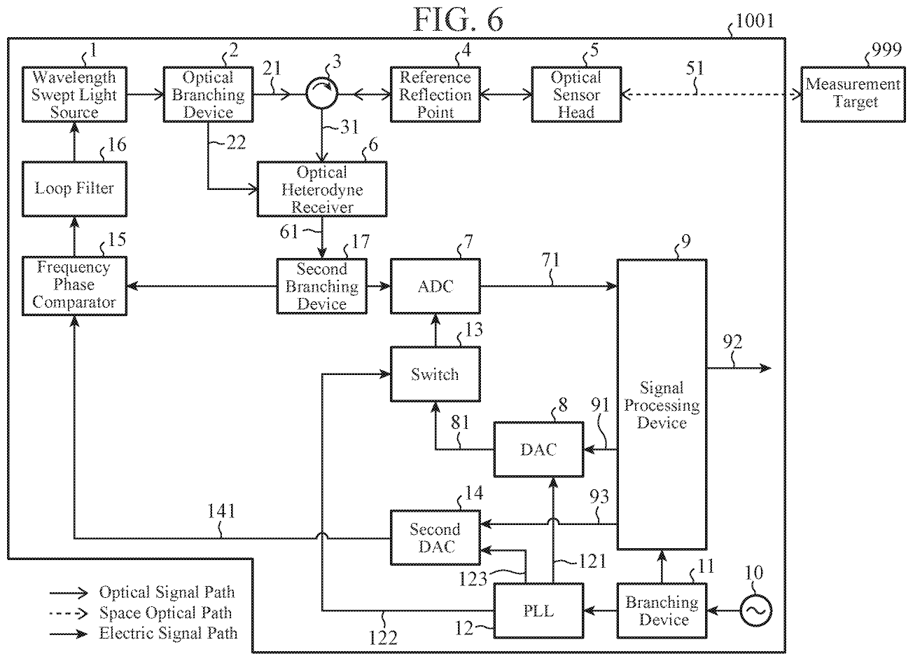

Hereinafter, the second embodiment will be described with reference to the drawings. Note that configurations having functions similar to those described in the first embodiment are denoted by the same reference numerals, and description thereof will be omitted. FIG. 6 is a block diagram illustrating a configuration of an optical sensor device 1001 according to the second embodiment. As illustrated in FIG. 6, the optical sensor device 1001 further includes a digital-to-analog converter 14 (second DAC) (second digital-to-analog converter), a frequency phase comparator 15, a loop filter 16, and a second branching device 17 (branching device) in addition to the configuration of the optical sensor device 1000 according to the first embodiment. Note that, in the second embodiment, as described above, it is assumed that the waveform of the wavelength swept light output from the wavelength swept light source 1 changes.

The second branching device 17 branches the internal reception signal acquired by the optical heterodyne receiver 6 into the frequency phase comparator 15 and the analog-to-digital converter 7. Note that, as described above, the internal reception signal here is acquired as an electric signal by the optical heterodyne receiver 6 multiplexing local oscillation light branched by the optical branching device 2 and internal reflected light reflected by the reference reflection point 4 and photoelectrically converting the multiplexed light. In the second embodiment, the optical heterodyne receiver 6 acquires an internal reception signal in a state where reflected light from the measurement target 999 is blocked.

The analog-to-digital converter 7 converts the internal reception signal branched by the second branching device 17 into a digital signal by sampling the internal reception signal in synchronization with the second clock signal generated by the phase-locked loop 12. The analog-to-digital converter 7 outputs the internal reception signal converted into the digital signal to the signal processing device 9. The signal processing device 9 further calculates second frequency variation reference signal data on the basis of the internal reception signal converted into a digital signal by the analog-to-digital converter 7. More specifically, in the second embodiment, the signal processing device 9 further calculates the second frequency variation reference signal data on the basis of the internal reception signal converted into a digital signal by the analog-to-digital converter 7 in synchronization with the reference clock signal branched by the branching device 11. The signal processing device 9 outputs the calculated second frequency variation reference signal data to the digital-to-analog converter 14 (93 in FIG. 6). More specifically, in the second embodiment, the signal processing device 9 stores the calculated second frequency variation reference signal data in a memory (not illustrated), and the memory outputs the stored second frequency variation reference signal data to the digital-to-analog converter 14.

The second frequency variation reference signal data may be, for example, an internal reception signal itself converted into a digital signal by the analog-to-digital converter 7. Alternatively, the signal processing device 9 may calculate the second frequency variation reference signal data by removing unnecessary frequency components from the internal reception signal converted into the digital signal by the analog-to-digital converter 7.

The digital-to-analog converter 14 generates a second frequency variation reference signal by converting the second frequency variation reference signal data calculated by the signal processing device 9 into an analog signal. More specifically, in the second embodiment, the digital-to-analog converter 14 generates the second frequency variation reference signal by converting the second frequency variation reference signal data calculated by the signal processing device 9 into an analog signal in synchronization with the second clock signal generated by the phase-locked loop 12. The digital-to-analog converter 14 outputs the generated second frequency variation reference signal to the frequency phase comparator 15 (141 in FIG. 1).

The frequency phase comparator 15 generates an error signal of frequency by comparing the internal reception signal branched by the second branching device 17 with the second frequency variation reference signal generated by the digital-to-analog converter 14. The frequency phase comparator 15 outputs the generated error signal to the loop filter 16.

The loop filter 16 generates a control signal by integrating the error signal generated by the frequency phase comparator 15. The loop filter 16 outputs the generated control signal to the wavelength swept light source 1.

The wavelength swept light source 1 adjusts the frequency of light to be output on the basis of the control signal generated by the loop filter 16.

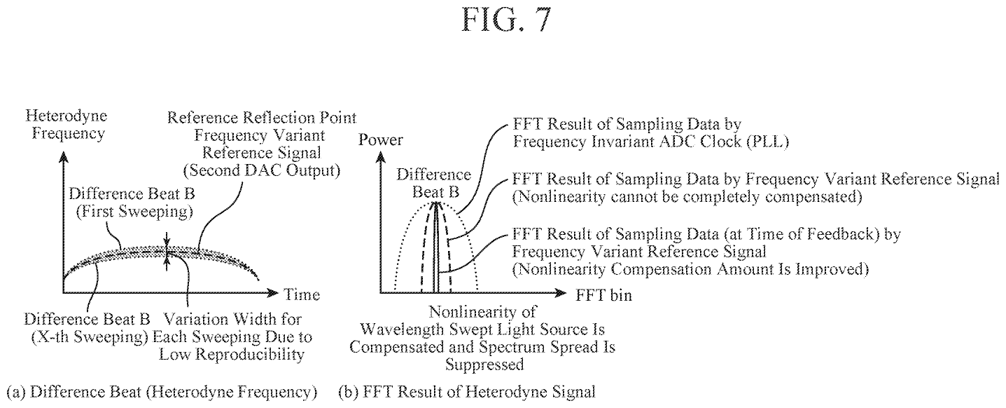

Hereinafter, a specific example of a method for compensating for nonlinearity of wavelength swept light by the optical sensor device 1001 according to the second embodiment will be described with reference to the drawings. FIG. 7 is a graph for describing a specific example of signal processing for internal reflected light by the optical sensor device 1001 according to the second embodiment. (a) of FIG. 7 is a graph illustrating a time change (dotted line) of a frequency (heterodyne frequency) of an internal reception signal (difference beat B) acquired by the optical heterodyne receiver 6 multiplexing the local oscillation light and the internal reflected light and photoelectrically converting the multiplexed light in the specific example. A broken line in (a) of FIG. 7 indicates the second frequency variation reference signal generated by the digital-to-analog converter 14.

As indicated by the dotted line in (a) of FIG. 7, the waveform of the wavelength swept light changes every time the wavelength swept light source 1 sweeps, so that the curve drawing the instantaneous frequency of the difference beat B changes. Therefore, at the time of a certain sweep, as described above, the frequency phase comparator 15 generates an error signal of frequency by comparing the difference beat B which is the internal reception signal branched by the second branching device 17 with the second frequency variation reference signal generated by the digital-to-analog converter 14. The loop filter 16 generates a control signal by integrating the error signal generated by the frequency phase comparator 15. The wavelength swept light source 1 adjusts the frequency of the light to be output on the basis of the control signal generated by the loop filter 16 to converge the frequency and phase of the wavelength swept light to be output to the same frequency and phase as those of the second reflection point frequency variation signal. Such a convergence operation improves the reproducibility of the nonlinearity of the wavelength swept light.

(b) of FIG. 7 is a graph illustrating a frequency spectrum (solid line) that is a result of the fast Fourier transform of the internal reception signal performed by the signal processing device 9 in the specific example. Note that the internal reception signal here is obtained as follows: the optical heterodyne receiver 6 acquires the internal reception signal derived from the wavelength swept light whose frequency has been adjusted on the basis of the control signal generated by the loop filter 16 by the wavelength swept light source 1, and the analog-to-digital converter 7 performs sampling in synchronization with the above-described first frequency variation reference signal to convert the internal reception signal into a digital signal. In addition, a dotted line in (b) of FIG. 7 indicates a frequency spectrum in a case where the analog-to-digital converter 7 converts the internal reception signal into a digital signal by sampling the internal reception signal in synchronization with the second clock signal of the phase-locked loop 12 described above. In addition, a broken line in (b) of FIG. 7 indicates a frequency spectrum when the wavelength swept light source 1 does not adjust the frequency of the wavelength swept light.

In a case where the wavelength swept light source 1 does not adjust the frequency of the wavelength swept light, nonlinearity of the wavelength swept light whose waveform changes is not compensated, so that the spectrum of the difference beat B spreads in the frequency axis direction as indicated by the broken line in (b) of FIG. 7. On the other hand, when the wavelength swept light source 1 adjusts the frequency of the wavelength swept light as described above, the nonlinearity of the wavelength swept light whose waveform changes is compensated, so that the spread of the spectrum of the difference beat B is suppressed as indicated by the solid line in (b) of FIG. 7.

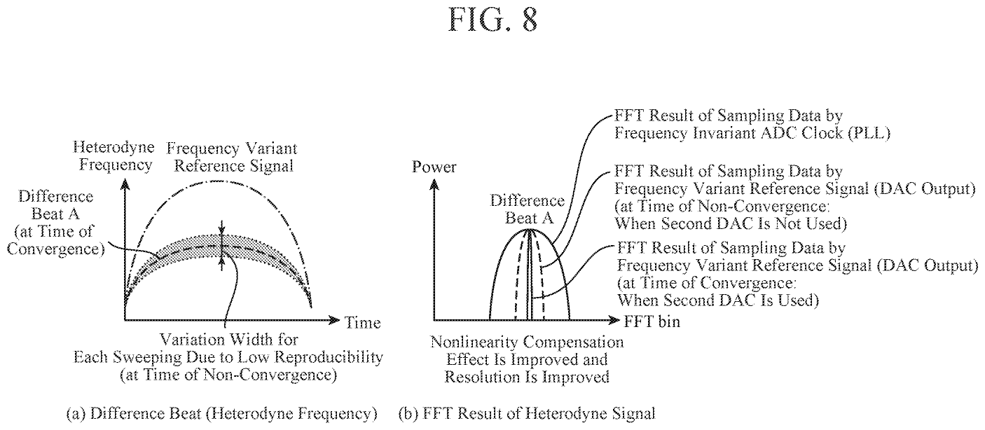

FIG. 8 is a graph for describing a specific example of signal processing for reflected light by the optical sensor device 1001 according to the second embodiment. (a) of FIG. 8 is a graph illustrating a time change (broken line) of the frequency (heterodyne frequency) of the reception signal (difference beat A) acquired by the optical heterodyne receiver 6 multiplexing the local oscillation light and the reflected light and photoelectrically converting the multiplexed light in the specific example. A dotted line in (a) of FIG. 8 is a graph illustrating a time change in the frequency of difference beat A when the wavelength swept light source 1 does not adjust the frequency of the wavelength swept light. An alternate long and short dash line in (a) of FIG. 8 indicates the first frequency variation reference signal.

As indicated by the dotted line in (a) of FIG. 8, the waveform of the wavelength swept light changes every time the wavelength swept light source 1 sweeps, so that the curve drawing the instantaneous frequency of the difference beat A changes. Therefore, the frequency and the phase of the wavelength swept light are converged to the same frequency and phase as those of the second reflection point frequency variation signal by adjusting the frequency of the light output from the wavelength swept light source 1 by the above-described means. As a result, as indicated by the broken line in (a) of FIG. 8, the instantaneous frequency of the difference beat A also converges, and the variation width for each sweep decreases.

(b) of FIG. 8 is a graph illustrating a frequency spectrum (inner solid line) that is a result of the fast Fourier transform of the reception signal performed by the signal processing device 9 in the specific example. Note that the reception signal here is acquired as follows: the optical heterodyne receiver 6 acquires the reception signal derived from the wavelength swept light whose frequency has been adjusted on the basis of the control signal generated by the loop filter 16 by the wavelength swept light source 1, and the analog-to-digital converter 7 performs sampling in synchronization with the above-described first frequency variation reference signal to convert the reception signal into a digital signal. Further, an outer solid line of (b) of FIG. 8 indicates a frequency spectrum in a case where the analog-to-digital converter 7 converts the reception signal into a digital signal by sampling the reception signal in synchronization with the second clock signal of the phase-locked loop 12 described above. In addition, a broken line in (b) of FIG. 8 indicates a frequency spectrum when the wavelength swept light source 1 does not adjust the frequency of the wavelength swept light.

In a case where the wavelength swept light source 1 does not adjust the frequency of the wavelength swept light, nonlinearity of the wavelength swept light whose waveform changes is not compensated, so that the spectrum of the difference beat A spreads in the frequency axis direction as indicated by the broken line in (b) of FIG. 8. On the other hand, when the wavelength swept light source 1 adjusts the frequency of the wavelength swept light as described above, the nonlinearity of the wavelength swept light whose waveform changes is compensated, so that the spread of the spectrum of the difference beat A is suppressed as indicated by the inner solid line of (b) of FIG. 8. As a result, the signal processing device 9 can calculate the position information of the measurement target on the basis of the FFT bin number.

As described above, in the second embodiment, the sensor resolution can be improved without using an additional interferometer for compensating for nonlinearity of the wavelength swept light whose waveform changes. In addition, it is possible to suppress nonlinearity of wavelength swept light due to environmental variations or the like and drift of measurement data due to a change in a swept frequency width, and for example, it is possible to improve measurement accuracy by averaging a plurality of times of measurement data.

### Third Embodiment

In a third embodiment, a configuration for separating a reception signal derived from reflected light reflected by the measurement target 999 and an internal reception signal derived from internal reflected light reflected by the reference reflection point 4 will be described.

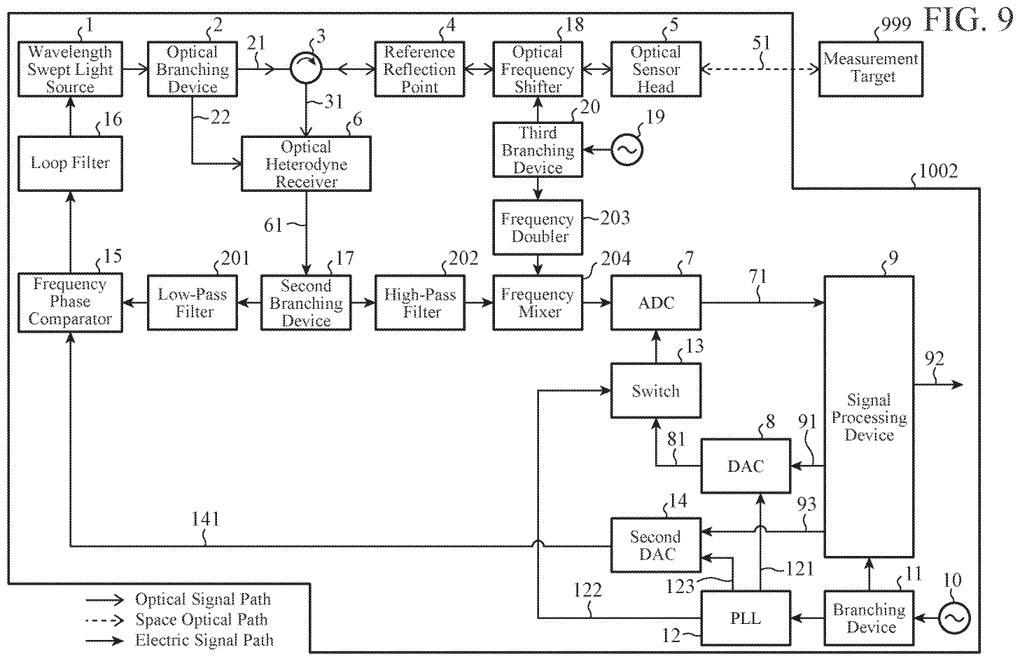

Hereinafter, the third embodiment will be described with reference to the drawings. Note that configurations having functions similar to those described in the first embodiment or the second embodiment are denoted by the same reference numerals, and description thereof will be omitted. FIG. 9 is a block diagram illustrating a configuration of an optical sensor device 1002 according to the third embodiment. As illustrated in FIG. 9, the optical sensor device 1002 includes an optical frequency shifter 18, a shift frequency oscillator 19, a third branching device 20, a low-pass filter 201 (first filter), a high-pass filter 202 (second filter), a frequency doubler 203, and a frequency mixer 204 in addition to the configuration of the optical sensor device 1001 according to the second embodiment. The optical frequency shifter 18 is installed between the reference reflection point 4 and the optical sensor head 5. The low-pass filter 201 is installed between the second branching device 17 and the frequency phase comparator 15. The high-pass filter 202 and the frequency mixer 204 are installed between the second branching device 17 and the analog-to-digital converter 7.

The shift frequency oscillator 19 outputs a frequency shift signal for performing frequency shift to the third branching device 20.

The third branching device 20 branches the frequency shift signal output from the shift frequency oscillator 19 into the optical frequency shifter 18 and the frequency doubler 203.

The frequency doubler 203 doubles the frequency shift signal branched by the third branching device 20. The frequency doubler 203 outputs the doubled frequency shift signal to the frequency mixer 204.

The optical frequency shifter 18 frequency-shifts the signal light having passed through the reference reflection point 4. More specifically, in the third embodiment, the optical frequency shifter 18 frequency-shifts the signal light having passed through the reference reflection point 4 on the basis of the frequency shift signal branched by the third branching device 20. More specifically, in the third embodiment, the optical frequency shifter 18 downshifts the frequency of the signal light having passed through the reference reflection point 4. The optical frequency shifter 18 outputs the frequency-shifted (downshifted) signal light to the optical sensor head 5.

As the optical frequency shifter 18, for example, an acousto-optical modulator (AOM) can be used. In this case, the waveform of the frequency shift signal output from the shift frequency oscillator 19 is a sin waveform. For example, as the optical frequency shifter 18, a LiNbO3 phase modulator that applies serrodyne modulation by applying a linear phase chirp to the signal light having passed through the reference reflection point 4 can be used. In that case, the waveform of the frequency shift signal output from the shift frequency oscillator 19 is a sawtooth waveform that repeats a linear voltage change.

The optical sensor head 5 emits the signal light frequency-shifted by the optical frequency shifter 18 toward the measurement target, and receives the reflected light reflected by the measurement target. The optical sensor head 5 outputs the received reflected light to the optical frequency shifter 18. The optical frequency shifter 18 frequency-shifts the reflected light output from the optical sensor head 5 again. The optical frequency shifter 18 outputs the frequency-shifted reflected light to the optical heterodyne receiver 6 via the reference reflection point 4 and the optical circulator 3.

The optical heterodyne receiver 6 multiplexes the local oscillation light branched by the optical branching device 2 and the reflected light output from the optical frequency shifter 18, and photoelectrically converts the multiplexed light to acquire a reception signal as an electric signal. In addition, the optical heterodyne receiver 6 multiplexes the local oscillation light branched by the optical branching device 2 and the internal reflected light reflected by the reference reflection point 4, and photoelectrically converts the multiplexed light to further acquire an internal reception signal as an electric signal.

The second branching device 17 branches the reception signal and the internal reception signal acquired by the optical heterodyne receiver 6 into a low-pass filter 201 and a high-pass filter 202.

The low-pass filter 201 passes the internal reception signal branched by the second branching device 17 and blocks the reception signal branched by the second branching device 17. That is, due to the downshifting by the optical frequency shifter 18 described above, the reception signal that is a beat signal based on the frequency difference between the reflected light and the local oscillation light has a higher frequency than the internal reception signal that is a beat signal based on the frequency difference between the internal reflected light and the local oscillation light, and thus is blocked by the low-pass filter 201.

The high-pass filter 202 passes the reception signal branched by the second branching device 17 and blocks the internal reception signal branched by the second branching device 17. That is, the reception signal that is a beat signal based on the frequency difference between the reflected light and the local oscillation light has a higher frequency than the internal reception signal that is a beat signal based on the frequency difference between the internal reflected light and the local oscillation light due to the downshifting by the optical frequency shifter 18 described above, and thus passes through the high-pass filter 202.

The frequency phase comparator 15 generates an error signal of frequency by comparing the internal reception signal passed by the low-pass filter 201 with the second frequency variation reference signal generated by the digital-to-analog converter 14.

The frequency mixer 204 frequency-shifts the reception signal passed by the high-pass filter 202 by a frequency that is twice the shift amount by the optical frequency shifter 18. More specifically, in the third embodiment, the frequency mixer 204 downshifts the reception signal by multiplying the reception signal passed by the high-pass filter 202 by the frequency shift signal doubled by the frequency doubler 203. The frequency mixer 204 outputs the frequency-shifted (downshifted) reception signal to the analog-to-digital converter 7.

The analog-to-digital converter 7 samples the reception signal passed by the high-pass filter 202 in synchronization with the first frequency variation reference signal generated in advance by the digital-to-analog converter 8. More specifically, in the third embodiment, the analog-to-digital converter 7 samples the reception signal frequency-shifted by the frequency mixer 204 in synchronization with the first frequency variation reference signal generated by the digital-to-analog converter 8.

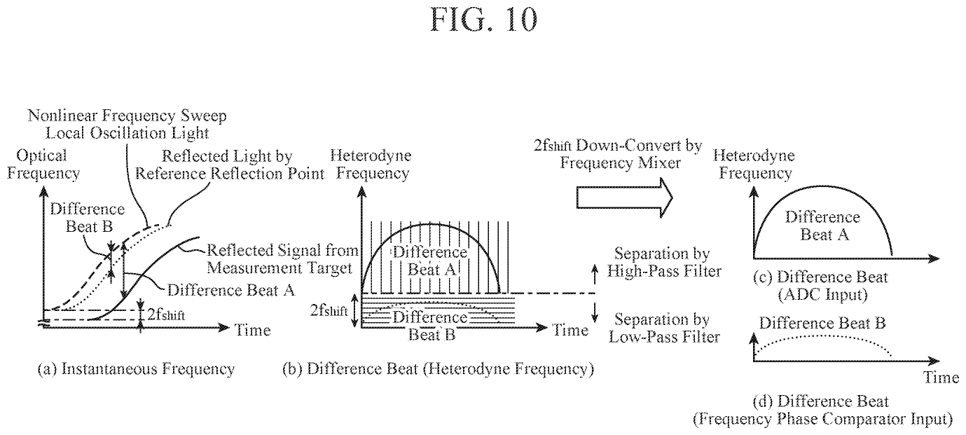

Hereinafter, a specific example of a method for separating a reception signal and an internal reception signal by the optical sensor device 1002 according to the third embodiment will be described. FIG. 10 is a graph illustrating a specific example of a method for separating a reception signal and an internal reception signal by the optical sensor device 1002 according to the third embodiment. (a) of FIG. 10 is a graph illustrating a time change (broken line) in the frequency of the local oscillation light branched by the optical branching device 2, a time change (dotted line) in the frequency of the internal reflected light reflected by the reference reflection point 4, and a time change (solid line) in the frequency of the reflected light received from the measurement target 999 by the optical sensor head 5 and frequency-shifted again by the optical frequency shifter 18 in the specific example.

The optical frequency shifter 18 frequency-shifts the signal light having passed through the reference reflection point 4 by an amount corresponding to f_{shift} (corresponding to the frequency of the shift frequency oscillator 19), and downshifts the frequency of the reflected light received from the measurement target 999 by the optical sensor head 5 again by an amount corresponding to f_{shift}. As a result, as illustrated in (a) of FIG. 10, only the reflected light component of the light input to the optical heterodyne receiver 6 undergoes a downshift of 2f_{shift}.

(b) of FIG. 10 is a graph illustrating a time change (dotted line) in the frequency (heterodyne frequency) of the internal reception signal (difference beat B) acquired by the optical heterodyne receiver 6 multiplexing the local oscillation light and the internal reflected light and photoelectrically converting the multiplexed light, and a time change (solid line) in the frequency (heterodyne frequency) of the reception signal (difference beat A) acquired by the optical heterodyne receiver 6 multiplexing the local oscillation light and the reflected light and photoelectrically converting the multiplexed light in the specific example.

For example, by setting f_{shift} so that 2f_{shift} is larger than the maximum value of the instantaneous frequency of the difference beat B between the local oscillation light and the internal reflected light by the reference reflection point 4 during the sweep, the difference beat A between the local oscillation light and the reflected light reflected by the measurement target can be selectively extracted by the high-pass filter 202 as illustrated in (b) of FIG. 10. As a result, the difference beat B can be selectively extracted by the low-pass filter 201.

(c) of FIG. 10 illustrates a time change in the frequency of the reception signal (difference beat A) input to the analog-to-digital converter 7. (d) of FIG. 10 illustrates a time change in the frequency of the internal reception signal (difference beat B) input to the frequency phase comparator 15.

As illustrated in (d) of FIG. 10, only the internal reception signal (difference beat B) of the reception signal and the internal reception signal branched by the second branching device 17 into the low-pass filter 201 is selectively extracted by the low-pass filter 201 and input to the frequency phase comparator 15. On the other hand, only the reception signal (difference beat A) of the reception signal and the internal reception signal branched by the second branching device 17 into the high-pass filter 202 is selectively extracted by the high-pass filter 202 and input to the frequency mixer 204. Then, the frequency mixer 204 down-converts the input reception signal by a frequency that is twice the shift amount by the optical frequency shifter 18. As a result, as illustrated in (c) of FIG. 10, the reception signal is input to the analog-to-digital converter 7 in a state where the shift component is removed by the optical frequency shifter 18.

As described above, in the third embodiment, in addition to the effects of the second embodiment, unnecessary reception signal components derived from reflected light from the measurement target can be removed from the signal input to the frequency phase comparator 15, convergence accuracy of the wavelength swept light can be improved, and resolution of position measurement of the measurement target can be improved.

Note that the signal processing device 9 may compensate for nonlinearity of the reception signal caused by the frequency shift by the optical frequency shifter 18 when calculating the measurement data related to the measurement target 999 on the basis of the reception signal converted into the digital signal by the analog-to-digital converter 7.

### Fourth Embodiment

In the second embodiment, the configuration in which the wavelength swept light source 1 compensates for nonlinearity of wavelength swept light whose waveform changes by adjusting the frequency of the wavelength swept light has been described. In a fourth embodiment, a configuration for compensating for nonlinearity of wavelength swept light whose waveform changes by frequency-shifting local oscillation light branched by the optical branching device 2 will be described.

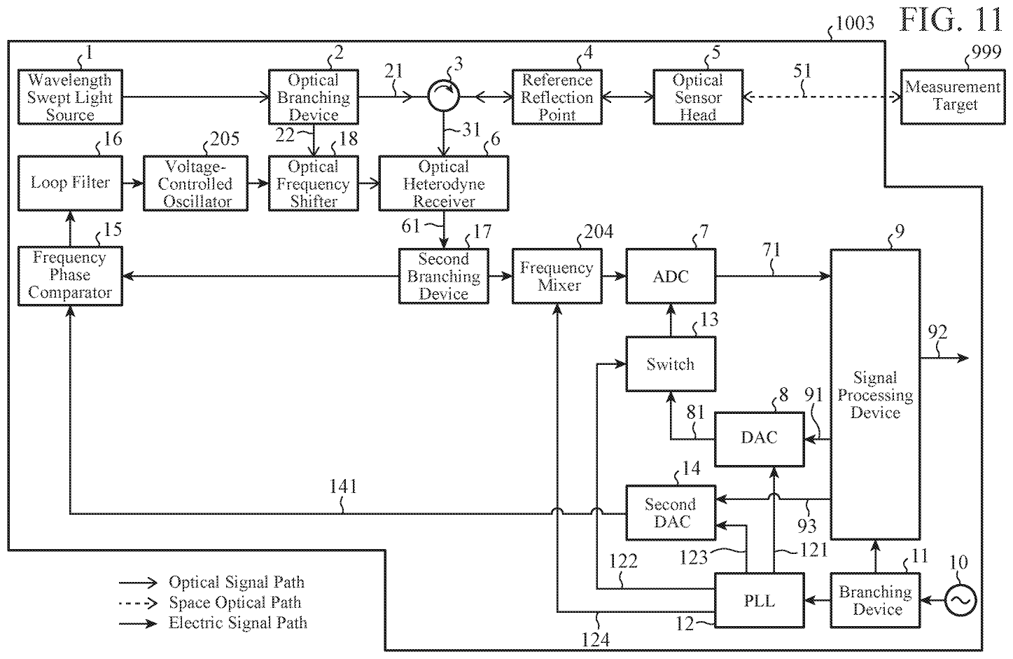

Hereinafter, the fourth embodiment will be described with reference to the drawings. Note that configurations having functions similar to those described in the first embodiment, the second embodiment, or the third embodiment are denoted by the same reference numerals, and description thereof will be omitted. FIG. 11 is a block diagram illustrating a configuration of an optical sensor device 1003 according to the fourth embodiment. As illustrated in FIG. 11, the optical sensor device 1003 further includes an optical frequency shifter 18, a frequency mixer 204, and a voltage-controlled oscillator 205 in addition to the configuration of the optical sensor device 1001 according to the second embodiment.

The loop filter 16 according to the fourth embodiment generates a control signal by integrating the error signal generated by the frequency phase comparator 15, and outputs the generated control signal to the voltage-controlled oscillator 205.

The voltage-controlled oscillator 205 generates a control signal of the optical frequency shifter 18 on the basis of the control signal generated by the loop filter 16. The voltage-controlled oscillator 205 outputs the generated control signal to the optical frequency shifter 18.

The optical frequency shifter 18 frequency-shifts the local oscillation light branched by the optical branching device 2 on the basis of the control signal generated by the voltage-controlled oscillator 205. The optical frequency shifter 18 outputs the frequency-shifted local oscillation light to the optical heterodyne receiver 6.

The optical heterodyne receiver 6 multiplexes the local oscillation light frequency-shifted by the optical frequency shifter 18 and the internal reflected light obtained by internally reflecting signal light branched by the optical branching device 2, and photoelectrically converts the multiplexed light to acquire an internal reception signal. More specifically, in the fourth embodiment, the optical heterodyne receiver 6 acquires an internal reception signal by multiplexing the local oscillation light frequency-shifted by the optical frequency shifter 18 and the internal reflected light reflected by the reference reflection point 4 and photoelectrically converting the multiplexed light.

When the optical sensor device 1003 measures the measurement data on the measurement target 999, the optical heterodyne receiver 6 multiplexes the local oscillation light frequency-shifted by the optical frequency shifter 18 and the reflected light received by the optical sensor head 5, and photoelectrically converts the multiplexed light to acquire a reception signal.

The frequency mixer 204 frequency-shifts the internal reception signal branched by the second branching device 17. When the optical sensor device 1003 measures the measurement data related to the measurement target 999, the frequency mixer 204 frequency-shifts the reception signal branched by the second branching device 17. More specifically, the frequency mixer 204 frequency-shifts the internal reception signal and the reception signal in synchronization with the second clock signal (124 in FIG. 11) generated by the phase-locked loop 12. A detailed configuration of the frequency mixer 204 will be described later.

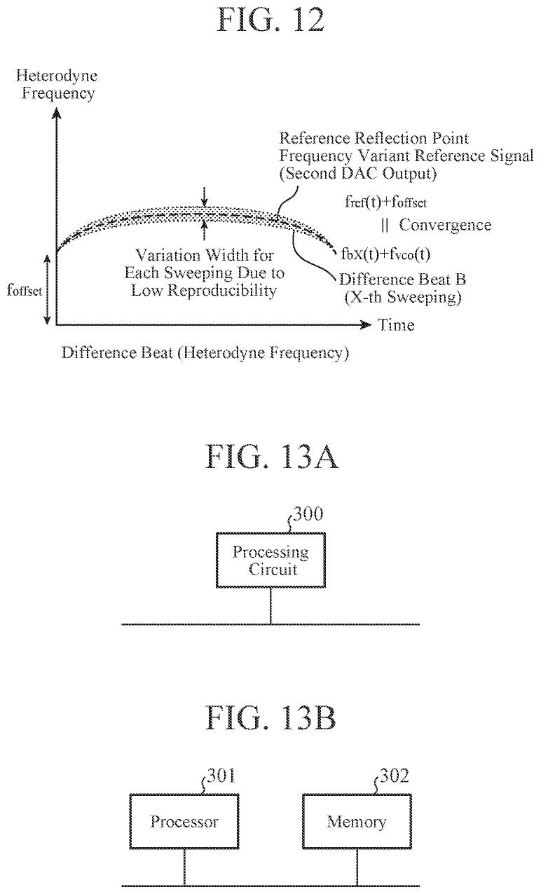

Hereinafter, a specific example of a method for compensating for nonlinearity of wavelength swept light by the optical sensor device 1003 according to the fourth embodiment will be described with reference to the drawings. FIG. 12 is a graph illustrating a time change (dotted line) in the frequency (heterodyne frequency) of the internal reception signal (difference beat B) acquired by the optical heterodyne receiver 6 multiplexing the local oscillation light and the internal reflected light and photoelectrically converting the multiplexed light in the specific example. A broken line in FIG. 12 indicates the second frequency variation reference signal generated by the digital-to-analog converter 14.

In this specific example, the optical frequency shifter 18 frequency-shifts the local oscillation light branched by the optical branching device 2 by an amount corresponding to the instantaneous frequency f_{vco}(t) on the basis of the control signal generated by the voltage-controlled oscillator 205. As a result, as indicated by the dotted line in FIG. 12, the instantaneous heterodyne frequency of the internal reception signal (difference beat B) acquired by the optical heterodyne receiver 6 at a certain sweep time X is f_{bx}(t)+f_{vco}(t). f_{bx}(t) is the frequency of the difference beat B at a certain sweep time X.

The signal processing device 9 calculates the second frequency variation reference signal data by giving an offset to the frequency of the internal reception signal converted into the digital signal by the analog-to-digital converter 7. More specifically, in the specific example, the signal processing device 9 gives the offset f_{offset} to the frequency of the internal reception signal converted into the digital signal by the analog-to-digital converter 7, thereby calculating the second frequency variation reference signal data having the frequency of f_{ref}(t)+f_{offset} as indicated by the broken line in FIG. 12. As a result, the control signal generated by the loop filter 16 on the basis of the error signal generated by the frequency phase comparator 15 controls the instantaneous frequency f_{vco}(t) of the control signal output by the voltage-controlled oscillator 205 so that f_{bx}(t)+f_{vco}(t)=f_{ref}(t)+f_{offset} holds.

The frequency mixer 204 downshifts the frequency of the internal reception signal branched by the second branching device 17 by an amount corresponding to the offset. More specifically, in the specific example, the frequency mixer 204 down-converts the frequency of the internal reception signal (difference beat B) branched by the second branching device 17 by an amount corresponding to the offset f_{offset}. As a result, the internal reception signal of the difference beat B converges to f_{bx}(t)+f_{vco}(t)−f_{offset}=f_{ref}(t). The analog-to-digital converter 7 samples the internal reception signal downshifted by the frequency mixer 204.

When measuring the measurement data related to the measurement target 999, the frequency mixer 204 downshifts the frequency of the reception signal branched by the second branching device 17 by an amount corresponding to the offset f_{offset}. The analog-to-digital converter 7 samples the reception signal downshifted by the frequency mixer 204 in synchronization with the first frequency variation reference signal generated by the digital-to-analog converter 8.

As described above, in the fourth embodiment, since the comparison frequency in the frequency phase comparator 15 can be increased by the amount corresponding to the offset, there is an effect that the operation is stabilized, and highly accurate measurement data can be obtained. In addition, since the nonlinearity of the wavelength swept light whose waveform changes can be compensated by frequency-shifting the local oscillation light, a wavelength swept light source that cannot externally control the wavelength sweep can be used, and the degree of freedom in design can be improved.

The function of the signal processing device 9 of the optical sensor device 1000, the optical sensor device 1001, the optical sensor device 1002, or the optical sensor device 1003 is implemented by a processing circuit. That is, the signal processing device 9 includes a processing circuit for executing the above-described processing. The processing circuit may be dedicated hardware, or may be a central processing unit (CPU) that executes a program stored in a memory.

FIG. 13A is a block diagram illustrating a hardware configuration that implements the functions of the signal processing device 9 of the optical sensor device 1000, the optical sensor device 1001, the optical sensor device 1002, or the optical sensor device 1003. FIG. 13B is a block diagram illustrating a hardware configuration that executes software for implementing the functions of the signal processing device 9 of the optical sensor device 1000, the optical sensor device 1001, the optical sensor device 1002, or the optical sensor device 1003.

In a case where the processing circuit is a processing circuit 300 of dedicated hardware illustrated in FIG. 13A, the processing circuit 300 corresponds to, for example, a single circuit, a composite circuit, a programmed processor, a parallel programmed processor, an application specific integrated circuit (ASIC), a field-programmable gate array (FPGA), or a combination thereof.

The functions of the signal processing device 9 of the optical sensor device 1000, the optical sensor device 1001, the optical sensor device 1002, or the optical sensor device 1003 may be implemented by separate processing circuits, or these functions may be collectively implemented by one processing circuit.

In a case where the processing circuit is a processor 301 illustrated in FIG. 13B, the functions of the signal processing device 9 of the optical sensor device 1000, the optical sensor device 1001, the optical sensor device 1002, or the optical sensor device 1003 are implemented by software, firmware, or a combination of software and firmware.

Note that the software or firmware is described as a program and stored in a memory 302.

The processor 301 reads and executes the program stored in the memory 302 to implement the functions of the signal processing device 9 of the optical sensor device 1000, the optical sensor device 1001, the optical sensor device 1002, or the optical sensor device 1003. That is, the signal processing device 9 of the optical sensor device 1000, the optical sensor device 1001, the optical sensor device 1002, or the optical sensor device 1003 includes the memory 302 for storing a program that results in execution of the above-described processing when each of these functions is executed by the processor 301.

These programs cause a computer to execute procedures or methods of the signal processing device 9 of the optical sensor device 1000, the optical sensor device 1001, the optical sensor device 1002, or the optical sensor device 1003. The memory 302 may be a computer-readable storage medium storing a program for causing a computer to function as the signal processing device 9 of the optical sensor device 1000, the optical sensor device 1001, the optical sensor device 1002, or the optical sensor device 1003.

The processor 301 corresponds to, for example, a central processing unit (CPU), a processing device, an arithmetic device, a processor, a microprocessor, a microcomputer, a digital signal processor (DSP), or the like.

The memory 302 corresponds to, for example, a nonvolatile or volatile semiconductor memory such as a random access memory (RAM), a read only memory (ROM), a flash memory, an erasable programmable read only memory (EPROM), or an electrically-EPROM (EEPROM), a magnetic disk such as a hard disk or a flexible disk, a flexible disk, an optical disk, a compact disk, a mini disk, a digital versatile disc (DVD), or the like.

The functions of the signal processing device 9 of the optical sensor device 1000, the optical sensor device 1001, the optical sensor device 1002, or the optical sensor device 1003 may be partially implemented by dedicated hardware, and partially implemented by software or firmware.

As described above, the processing circuit can implement each of the above-described functions by hardware, software, firmware, or a combination thereof.

Note that it is possible to freely combine the individual embodiments, to modify any components of the individual embodiments, or to omit any components in the individual embodiments.

### INDUSTRIAL APPLICABILITY

The optical sensor device according to the present disclosure can reduce a signal processing load caused by compensating for nonlinearity of the wavelength swept light, and thus can be used for a technology of compensating for nonlinearity of the wavelength swept light.

### REFERENCE SIGNS LIST

1: wavelength swept light source, 2: optical branching device, 3: optical circulator, 4: reference reflection point, 5: optical sensor head, 6: optical heterodyne receiver, 7: analog-to-digital converter, 8: digital-to-analog converter, 9: signal processing device, 10: reference clock, 11: branching device, 12: phase-locked loop, 13: switch, 14: digital-to-analog converter, 15: frequency phase comparator, 16: loop filter, 17: second branching device, 18: optical frequency shifter, 19: shift frequency oscillator, 20: third branching device, 201: low-pass filter, 202: high-pass filter, 203: frequency doubler, 204: frequency mixer, 205: voltage-controlled oscillator, 300: processing circuit, 301: processor, 302: memory, 999: measurement target, 1000, 1001, 1002, 1003: optical sensor device

1. An optical sensor device, comprising:
a wavelength swept light source to output light whose frequency changes with lapse of time;
an optical brancher to branch light output from the wavelength swept light source into signal light and local oscillation light;
an optical sensor head to emit the signal light branched by the optical brancher toward a measurement target and receive reflected light reflected by the measurement target;
an optical heterodyne receiver to multiplex the local oscillation light branched by the optical brancher and the reflected light received by the optical sensor head, and photoelectrically convert the multiplexed light to acquire a reception signal as an electric signal;
an analog-to-digital converter to convert the reception signal acquired by the optical heterodyne receiver into a digital signal by sampling the reception signal;
a first digital-to-analog converter to generate a first clock signal of the analog-to-digital converter;
a phase-locked loop to generate a second clock signal of the analog-to-digital converter; and
a signal processor to calculate measurement data related to the measurement target on a basis of the reception signal converted into the digital signal by the analog-to-digital converter, wherein
the optical heterodyne receiver multiplexes the local oscillation light branched by the optical brancher and internal reflected light obtained by internally reflecting the signal light branched by the optical brancher, and photoelectrically converts the multiplexed light to further acquire an internal reception signal as an electric signal,
the analog-to-digital converter further converts the internal reception signal acquired by the optical heterodyne receiver into a digital signal by sampling the internal reception signal,
the signal processor further calculates first frequency variation reference signal data serving as a reference for frequency variation of light output from the wavelength swept light source on a basis of the internal reception signal converted into a digital signal by the analog-to-digital converter,
the first digital-to-analog converter generates a first frequency variation reference signal as the first clock signal by converting the first frequency variation reference signal data calculated by the signal processor into an analog signal,
the analog-to-digital converter samples the reception signal acquired by the optical heterodyne receiver in synchronization with the first frequency variation reference signal generated by the first digital-to-analog converter or,
samples the internal reception signal acquired by the optical heterodyne receiver in synchronization with the second clock signal generated by the phase-locked loop.

2. An optical sensor device, comprising:
a wavelength swept light source to output light whose frequency changes with lapse of time;
an optical brancher to branch light output from the wavelength swept light source into signal light and local oscillation light;
an optical sensor head to emit the signal light branched by the optical brancher toward a measurement target and receive reflected light reflected by the measurement target;
an optical heterodyne receiver to multiplex the local oscillation light branched by the optical brancher and the reflected light received by the optical sensor head, and photoelectrically convert the multiplexed light to acquire a reception signal as an electric signal;
an analog-to-digital converter to convert the reception signal acquired by the optical heterodyne receiver into a digital signal by sampling the reception signal;
a first digital-to-analog converter to generate a first clock signal of the analog-to-digital converter; and
a signal processor to calculate measurement data related to the measurement target on a basis of the reception signal converted into the digital signal by the analog-to-digital converter, wherein
the optical heterodyne receiver multiplexes the local oscillation light branched by the optical brancher and internal reflected light obtained by internally reflecting the signal light branched by the optical brancher, and photoelectrically converts the multiplexed light to further acquire an internal reception signal as an electric signal,
the analog-to-digital converter further converts the internal reception signal acquired by the optical heterodyne receiver into a digital signal by sampling the internal reception signal,
the signal processor further calculates an instantaneous frequency of the internal reception signal by performing Hilbert transform on the internal reception signal converted into the digital signal by the analog-to-digital converter, and calculates first frequency variation reference signal data as a reference to frequency variation of light output by the wavelength swept light source by multiplying the calculated instantaneous frequency,
the first digital-to-analog converter generates a first frequency variation reference signal as the first clock signal by converting the first frequency variation reference signal data calculated by the signal processor into an analog signal, and
the analog-to-digital converter samples the reception signal acquired by the optical heterodyne receiver in synchronization with the first frequency variation reference signal generated by the first digital-to-analog converter.

3. An optical sensor device, comprising:
a wavelength swept light source to output light whose frequency changes with lapse of time;
an optical brancher to branch light output from the wavelength swept light source into signal light and local oscillation light;
an optical sensor head to emit the signal light branched by the optical brancher toward a measurement target and receive reflected light reflected by the measurement target;
an optical heterodyne receiver to multiplex the local oscillation light branched by the optical brancher and the reflected light received by the optical sensor head, and photoelectrically convert the multiplexed light to acquire a reception signal as an electric signal;
an analog-to-digital converter to convert the reception signal acquired by the optical heterodyne receiver into a digital signal by sampling the reception signal;
a first digital-to-analog converter to generate a first clock signal of the analog-to-digital converter;
a signal processor to calculate measurement data related to the measurement target on a basis of the reception signal converted into the digital signal by the analog-to-digital converter,
a brancher;
a second digital-to-analog converter;
a frequency phase comparator; and
a loop filter, wherein
the optical heterodyne receiver multiplexes the local oscillation light branched by the optical brancher and internal reflected light obtained by internally reflecting the signal light branched by the optical brancher, and photoelectrically converts the multiplexed light to further acquire an internal reception signal as an electric signal,
the analog-to-digital converter further converts the internal reception signal acquired by the optical heterodyne receiver into a digital signal by sampling the internal reception signal,
the signal processor further calculates first frequency variation reference signal data serving as a reference for frequency variation of light output from the wavelength swept light source on a basis of the internal reception signal converted into a digital signal by the analog-to-digital converter,
the first digital-to-analog converter generates a first frequency variation reference signal as the first clock signal by converting the first frequency variation reference signal data calculated by the signal processor into an analog signal,
the analog-to-digital converter samples the reception signal acquired by the optical heterodyne receiver in synchronization with the first frequency variation reference signal generated by the first digital-to-analog converter,
the brancher branches the internal reception signal acquired by the optical heterodyne receiver into the frequency phase comparator and the analog-to-digital converter,
the signal processor further calculates second frequency variation reference signal data on a basis of the internal reception signal converted into the digital signal by the analog-to-digital converter,
the second digital-to-analog converter generates a second frequency variation reference signal by converting the second frequency variation reference signal data calculated by the signal processor into an analog signal,
the frequency phase comparator generates an error signal of frequency by comparing the internal reception signal branched by the brancher with the second frequency variation reference signal generated by the second digital-to-analog converter,
the loop filter generates a control signal by integrating the error signal generated by the frequency phase comparator, and
the wavelength swept light source adjusts a frequency of light to be output on a basis of the control signal generated by the loop filter.

4. An optical sensor device, comprising:
a wavelength swept light source to output light whose frequency changes with lapse of time;
an optical brancher to branch light output from the wavelength swept light source into signal light and local oscillation light;
an optical sensor head to emit the signal light branched by the optical brancher toward a measurement target and receive reflected light reflected by the measurement target;
an optical heterodyne receiver to multiplex the local oscillation light branched by the optical brancher and the reflected light received by the optical sensor head, and photoelectrically convert the multiplexed light to acquire a reception signal as an electric signal;
an analog-to-digital converter to convert the reception signal acquired by the optical heterodyne receiver into a digital signal by sampling the reception signal;
a first digital-to-analog converter to generate a first clock signal of the analog-to-digital converter;
a signal processor to calculate measurement data related to the measurement target on a basis of the reception signal converted into the digital signal by the analog-to-digital converter;
a brancher;
a second digital-to-analog converter;
a frequency phase comparator;
a loop filter;
a voltage-controlled oscillator; and
an optical frequency shifter,
wherein
the optical heterodyne receiver multiplexes the local oscillation light branched by the optical brancher and internal reflected light obtained by internally reflecting the signal light branched by the optical brancher, and photoelectrically converts the multiplexed light to further acquire an internal reception signal as an electric signal,
the analog-to-digital converter further converts the internal reception signal acquired by the optical heterodyne receiver into a digital signal by sampling the internal reception signal,
the signal processor further calculates first frequency variation reference signal data serving as a reference for frequency variation of light output from the wavelength swept light source on a basis of the internal reception signal converted into a digital signal by the analog-to-digital converter,
the first digital-to-analog converter generates a first frequency variation reference signal as the first clock signal by converting the first frequency variation reference signal data calculated by the signal processor into an analog signal,
the analog-to-digital converter samples the reception signal acquired by the optical heterodyne receiver in synchronization with the first frequency variation reference signal generated by the first digital-to-analog converter,
the brancher branches the internal reception signal acquired by the optical heterodyne receiver into the frequency phase comparator and the analog-to-digital converter,
the signal processor further calculates second frequency variation reference signal data on a basis of the internal reception signal converted into the digital signal by the analog-to-digital converter,
the second digital-to-analog converter generates a second frequency variation reference signal by converting the second frequency variation reference signal data calculated by the signal processor into an analog signal,
the frequency phase comparator generates an error signal of frequency by comparing the internal reception signal branched by the brancher with the second frequency variation reference signal generated by the second digital-to-analog converter,
the loop filter generates a control signal by integrating the error signal generated by the frequency phase comparator,
the voltage-controlled oscillator generates a control signal of the optical frequency shifter on a basis of the control signal generated by the loop filter,
the optical frequency shifter frequency-shifts the local oscillation light branched by the optical brancher on a basis of the control signal generated by the voltage-controlled oscillator, and
the optical heterodyne receiver multiplexes the local oscillation light frequency-shifted by the optical frequency shifter and the internal reflected light obtained by internally reflecting the signal light branched by the optical brancher, photoelectrically converts the multiplexed light to acquire a internal reception signal, multiplexes the local oscillation light frequency-shifted by the optical frequency shifter and the reflected light received by the optical sensor head, and photoelectrically converts the multiplexed light to acquire a reception signal.

5. The optical sensor device according to claim 1, further comprising a reference reflection point to internally reflects the signal light branched by the optical brancher by partially reflecting the signal light, wherein
the optical heterodyne receiver multiplexes the local oscillation light branched by the optical brancher and the internal reflected light reflected by the reference reflection point, and photoelectrically converts the multiplexed light to further acquire an internal reception signal as an electric signal.

6. The optical sensor device according to claim 2, further comprising a reference reflection point to internally reflects the signal light branched by the optical brancher by partially reflecting the signal light, wherein
the optical heterodyne receiver multiplexes the local oscillation light branched by the optical brancher and the internal reflected light reflected by the reference reflection point, and photoelectrically converts the multiplexed light to further acquire an internal reception signal as an electric signal.

7. The optical sensor device according to claim 3, further comprising a reference reflection point to internally reflects the signal light branched by the optical brancher by partially reflecting the signal light, wherein
the optical heterodyne receiver multiplexes the local oscillation light branched by the optical brancher and the internal reflected light reflected by the reference reflection point, and photoelectrically converts the multiplexed light to further acquire an internal reception signal as an electric signal.

8. The optical sensor device according to claim 4, further comprising a reference reflection point to internally reflects the signal light branched by the optical brancher by partially reflecting the signal light, wherein
the optical heterodyne receiver multiplexes the local oscillation light branched by the optical brancher and the internal reflected light reflected by the reference reflection point, and photoelectrically converts the multiplexed light to further acquire an internal reception signal as an electric signal.

9. The optical sensor device according to claim 1, further comprising a switch to switch a clock signal of the analog-to-digital converter to either the first frequency variation reference signal as the first clock signal generated by the first digital-to-analog converter or the second clock signal generated by the phase-locked loop.

10. The optical sensor device according to claim 3, further comprising a reference reflection point, an optical frequency shifter, a first filter, and a second filter, wherein
the reference reflection point internally reflects the signal light branched by the optical brancher by partially reflecting the signal light,
the optical frequency shifter frequency-shifts the signal light having passed through the reference reflection point,
the optical sensor head emits the signal light frequency-shifted by the optical frequency shifter toward the measurement target and receives reflected light reflected by the measurement target,
the optical heterodyne receiver multiplexes the local oscillation light branched by the optical brancher and the internal reflected light reflected by the reference reflection point, and photoelectrically converts the multiplexed light to further acquire an internal reception signal as an electric signal,
the brancher branches the reception signal and the internal reception signal acquired by the optical heterodyne receiver into the first filter and the second filter,
the first filter passes the internal reception signal branched by the brancher and blocks the reception signal branched by the brancher,
the second filter passes the reception signal branched by the brancher and blocks the internal reception signal branched by the brancher,
the frequency phase comparator generates an error signal of frequency by comparing the internal reception signal passed by the first filter with the second frequency variation reference signal generated by the second digital-to-analog converter, and
the analog-to-digital converter samples the reception signal passed by the second filter in synchronization with the first frequency variation reference signal generated by the first digital-to-analog converter.

11. The optical sensor device according to claim 10, further comprising a frequency mixer, wherein
the optical frequency shifter further frequency-shifts the reflected light received by the optical sensor head,
the frequency mixer frequency-shifts the reception signal passed by the second filter by a frequency that is twice the shift amount by the optical frequency shifter, and
the analog-to-digital converter samples the reception signal frequency-shifted by the frequency mixer in synchronization with the first frequency variation reference signal generated by the first digital-to-analog converter.

12. The optical sensor device according to claim 10, wherein the signal processor compensates for nonlinearity of the reception signal caused by the frequency shift by the optical frequency shifter when calculating the measurement data related to the measurement target on a basis of the reception signal converted into the digital signal by the analog-to-digital converter.

13. The optical sensor device according to claim 4, further comprising a frequency mixer, wherein
the signal processor calculates the second frequency variation reference signal data by giving an offset to a frequency of the internal reception signal converted into the digital signal by the analog-to-digital converter, and
the frequency mixer downshifts each frequency of the reception signal and the internal reception signal branched by the brancher by an amount corresponding to the offset.
